(19) United States
(12) Patent Application Publication
(10) Pub. No.: US 2006/0116369 A1
(43) Pub. Date: Jun. 1, 2006

(54) Certain compounds, compositions, and methods

(76) Inventors: Shyamlal Ramchandani, San Francisco, CA (US); Reginald Norman De La Rosa, Concord, CA (US); Cynthia L. Adams, San Carlos, CA (US); Gustave Bergnes, Pacifica, CA (US); David J. Morgans JR., Los Altos, CA (US); Jay K. Trautman, Los Altos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/257,751

(22) Filed: Oct. 24, 2005

Related U.S. Application Data
(60) Provisional application No. 60/622,282, filed on Oct. 25, 2004.

(51) Int. Cl. A61K 31/541; A61K 31/5377; A61K 31/496; A61K 31/4709; C07D 417/02

(52) U.S. Cl. 514/227.8; 514/314; 546/167; 546/169; 514/232.5; 514/253.06; 544/60; 546/128; 544/363

(57) ABSTRACT

Compounds useful for treating cellular proliferative diseases are disclosed.

This application claims the benefit of U.S. Provisional Patent Application No. 60/622,282, filed Oct. 25, 2004, which is incorporated herein by reference for all purposes.

Provided are compounds that are useful in the treatment of cellular proliferative diseases, for example cancer, hyperplasias, restenosis, cardiac hypertrophy, immune disorders, fungal disorders, and inflammation.

Improvements in the specificity of agents used to treat cancer are of considerable interest. Reducing the side effects associated with the administration of these agents would result in significant therapeutic benefits. Traditionally, dramatic improvements in the treatment of cancer have been associated with identification of therapeutic agents acting through novel mechanisms. Examples of such agents include not only the taxanes, but also the camptothecin class of topoisomerase I inhibitors.

Provided is at least one chemical entity chosen from compounds of Formula I 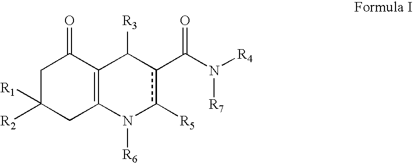
and pharmaceutically acceptable salts, solvates, chelates, non-covalent complexes, prodrugs, and mixtures thereof, wherein
R₁ and R₂ are independently selected from hydrogen, optionally substituted lower alkyl, optionally substituted cycloalkyl, optionally substituted heterocycloalkyl, optionally substituted aryl, and optionally substituted heteroaryl, or wherein R₁ taken together with R₂ form an optionally substituted 3- to 7-membered ring which optionally includes one or two heteroatoms chosen from O, N, and S;
R₃ is selected from optionally substituted aryl and optionally substituted heteroaryl;
R₄ is selected from hydrogen, optionally substituted lower alkyl, optionally substituted aryl and optionally substituted heteroaryl;
R₅ is chosen from hydrogen, optionally substituted lower alkyl, optionally substituted aryl, and optionally substituted heteroaryl;
R₆ is chosen from hydrogen and optionally substituted lower alkyl, or R₅ taken together with R₆, and the atoms to which they are bound form an optionally substituted 4 to 7-membered ring which optionally includes one, two, or three heteroatoms chosen from N, O, and S, and
R₇ is chosen from hydrogen and optionally substituted lower alkyl, or
R₄ and R₇, taken together with the nitrogen to which they are bound form an optionally substituted 4 to 7-membered ring which optionally includes one, two, or three heteroatoms chosen from N, O, and S, and
   wherein the dashed line indicates that the bond can be either a single or double bond.

Also provided is a pharmaceutical composition comprising a therapeutically effective amount of at least one chemical entity described herein and one or more pharmaceutical excipients.

Also provided is a method of treating a cellular proliferative disease comprising administering to a patient in need of such treatment at least one chemical entity described herein in a therapeutically effective amount to treat the cellular proliferative disease.

As used in the present specification, the following words and phrases are generally intended to have the meanings as set forth below, except to the extent that the context in which they are used indicates otherwise.

As used herein, when any variable occurs more than one time in a chemical formula, its definition on each occurrence is independent of its definition at every other occurrence. In accordance with the usual meaning of “a” and “the” in patents, reference, for example, to “a” kinase or “the” kinase is inclusive of one or more kinases.

Formula I includes all subformulae thereof. For example Formula I includes compounds of Formula II.

A dash (“−”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group. When an alkyl residue having a specific number of carbons is named, all geometric combinations having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having one to four carbons.

“Alkenyl” refers to an unsaturated branched or straight-chain alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkynyl” refers to an unsaturated branched or straight-chain alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms.

“Cycloalkyl” indicates a non-aromatic carbocyclic ring, usually having from 3 to 7 ring carbon atoms. The ring may be saturated or have one or more carbon-carbon double bonds. Examples of cycloalkyl groups include cyclopropyl, cyclobutyl, cyclopentyl, cyclopentenyl, cyclohexyl, and cyclohexenyl, as well as bridged and caged saturated ring groups such as norbornane.

By “alkoxy” is meant an alkyl group of the indicated number of carbon atoms attached through an oxygen bridge such as, for example, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, 3-methylpentyloxy, and the like. Alkoxy groups will usually have from 1 to 6 carbon atoms attached through the oxygen bridge. “Lower alkoxy” refers to alkoxy groups having one to four carbons.

“Mono- and di-alkylcarboxamide” encompasses a group of the formula—(C═O)NR^{a}R^{b} where R^{a} and R^{b} are independently chosen from hydrogen and alkyl groups of the indicated number of carbon atoms, provided that R^{a} and R^{b} are not both hydrogen.

“Acyl” refers to the groups (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

By “alkoxycarbonyl” is meant a group of the formula (alkoxy)(C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker.

By “amino” is meant the group —NH₂.

“Mono- and di-(alkyl)amino” encompasses secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

The term “aminocarbonyl” refers to the group —CONR^{b}R^{c}, where

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c} taken together with the nitrogen to which they are bound, form an optionally substituted 5- to 7-membered nitrogen-containing heterocycloalkyl which optionally includes 1 or 2 additional heteroatoms selected from O, N, and S in the heterocycloalkyl ring;
where each substituted group is independently substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C—C₄ alkyl-OH, —OC—C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

“Aryl” encompasses:
6-membered carbocyclic aromatic rings, for example, benzene;
bicyclic ring systems wherein at least one ring is carbocyclic and aromatic, for example, naphthalene, indane, and tetralin; and
tricyclic ring systems wherein at least one ring is carbocyclic and aromatic, for example, fluorene.
   Aryl includes 6-membered carbocyclic aromatic rings fused to a 5- to 7-membered heterocycloalkyl ring containing 1 or more heteroatoms chosen from N, O, and S. For example, aryl includes phenyl substituted with —O(C₁-C₂ alkyl)O— (e.g., phenyl substituted with a methylenedioxy or ethylenedioxy group). For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

The term “aryloxy” refers to the group —O-aryl.

The term “halo” includes fluoro, chloro, bromo, and iodo, and the term “halogen” includes fluorine, chlorine, bromine, and iodine.

“Haloalkyl” indicates alkyl as defined above having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

“Heteroaryl” encompasses:
5- to 7-membered aromatic, monocyclic rings containing one or more, for example, from 1 to 4, or in certain embodiments, from 1 to 3, heteroatoms chosen from N, O, and S, with the remaining ring atoms being carbon; and
bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring.
   Heteroaryl includes a 5- to 7-membered heterocycloalkyl, aromatic ring fused to a 5- to 7-membered cycloalkyl or heterocycloalkyl ring. For example, heteroaryl includes pyridinyl substituted with —O(C₁-C₂ alkyl)O— (e.g., pyridinyl substituted with a methylenedioxy or ethylenedioxy group). For such fused, bicyclic heteroaryl ring systems, the point of attachment may be at either ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridazinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl, cycloalkyl, or heterocycloalkyl, as defined herein

Substituted heteroaryl also includes ring systems substituted with one or more oxide (—O⁻) substituents, such as pyridinyl N-oxides.

By “heterocycloalkyl” is meant a single, non-aromatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. The ring may be saturated or have one or more carbon-carbon double bonds. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperdyl, and 2,5-piperzinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (═O) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteratoms independently selected from oxygen, sulfur, and nitrogen and is not-aromatic.

As used herein, “modulation” refers to a change in kinase activity as a direct or indirect response to the presence of compounds of Formula I, relative to the activity of the kinase in the absence of the compound. The change may be an increase in activity or a decrease in activity, and may be due to the direct interaction of the compound with the kinase, or due to the interaction of the compound with one or more other factors that in turn affect kinase activity. For example, the presence of the compound may, for example, increase or decrease kinase activity by directly binding to the kinase, by causing (directly or indirectly) another factor to increase or decrease the kinase activity, or by (directly or indirectly) increasing or decreasing the amount of kinase present in the cell or organism.

The term “sulfanyl” includes the groups: —S-(optionally substituted (C₁-C₆)alkyl), —S-(optionally substituted aryl), —S-(optionally substituted heteroaryl), and —S-(optionally substituted heterocycloalkyl). Hence, sulfanyl includes the group C₁-C₆ alkylsulfanyl.

The term “sulfinyl” includes the groups: —S(O)-(optionally substituted (C₁-C₆)alkyl), —S(O)-optionally substituted aryl), —S(O)-optionally substituted heteroaryl), —S(O)-(optionally substituted heterocycloalkyl); and —S(O)-(optionally substituted amino).

The term “sulfonyl” includes the groups: —S(O₂)-(optionally substituted (C₁-C₆)alkyl), —S(O₂)-optionally substituted aryl), —S(O₂)-optionally substituted heteroaryl), —S(O₂)-(optionally substituted heterocycloalkyl), —S(O₂)-(optionally substituted alkoxy), —S(O₂)-optionally substituted aryloxy), —S(O₂)-optionally substituted heteroaryloxy), —S(O₂)-(optionally substituted heterocyclyloxy); and —S(O₂)-(optionally substituted amino).

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (such as up to 5, for example, up to 3) hydrogen atoms are replaced by a substituent independently chosen from:

-R^{a}, —OR^{b}, —SR^{b}, guanidine, guanidine wherein one or more of the guanidine hydrogens are replaced with a lower-alkyl group, —NR^{b}R^{c}, halo, cyano, nitro, oxo (as a substitutent for cycloalkyl, heterocycloalkyl, and heteroaryl), —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted acyl” refers to the groups (substituted alkyl)-C(O)—; (substituted cycloalkyl)-C(O)—; (substituted aryl)-C(O)—; (substituted heteroaryl)-C(O)—; and (substituted heterocycloalkyl)-C(O)—, wherein the group is attached to the parent structure through the carbonyl functionality and wherein substituted alkyl, cycloalkyl, aryl, heteroaryl, and heterocycloalkyl, refer respectively to alkyl, cycloalkyl, aryl, heteroaryl, and heterocycloalkyl wherein one or more (such as up to 5, for example, up to 3) hydrogen atoms are replaced by a substituent independently chosen from:

-R^{a}, —OR^{b}, —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted alkoxy” refers to alkoxy wherein the alkyl constituent is substituted (i.e., —O-(substituted alkyl)) wherein “substituted alkyl” refers to alkyl wherein one or more (such as up to 5, for example, up to 3) hydrogen atoms are replaced by a substituent independently chosen from:

-R^{a}, —OR^{b}, —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO²R^{a}, —SO₂NR^{b}R^{c}, and —NR^{c}SO₂R^{a},

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₙCH₃, where x is an integer of 2-20, such as 2-10, and for example, 2-5. Another substituted alkoxy group is hydroxyalkoxy or —OCH₂(CH₂)_{y}OH, where y is an integer of 1-10, such as 1-4.

The term “substituted alkoxycarbonyl” refers to the group (substituted alkyl)-O—C(O)— wherein the group is attached to the parent structure through the carbonyl functionality and wherein substituted refers to alkyl wherein one or more (such as up to 5, for example, up to 3) hydrogen atoms are replaced by a substituent independently chosen from:

—R^{a}, —OR^{b}, —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OC₂R^{a}, —OCONR^{b}R^{c}, —NR^{c}COR, —NRcCO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted amino” refers to the group —NHR^{d} or —NR^{d}R^{e} wherein R^{d} is chosen from: hydroxy, optionally substitued alkoxy, optionally substituted alkyl, optionally substituted cycloalkyl, optionally substituted acyl, optionally substituted aminocarbonyl, optionally substituted aryl, optionally substituted heteroaryl, optionally substituted heterocycloalkyl, optionally substituted alkoxycarbonyl, sulfinyl and sulfonyl, and wherein R^{e} is chosen from: optionally substituted alkyl, optionally substituted cycloalkyl, optionally substituted aryl, optionally substituted heteroaryl, and optionally substituted heterocycloalkyl, and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently chosen from:

-R^{a}, —OR^{b}, —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NRcCO₂R^{a}, —NR^{c}CONR^{b}R^{c}, —CO₂R^{b}, —CONR^{b}R^{c}, —NR^{c}COR^{b}, SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted alkenyl, optionally substituted alkynyl, optionally substituted aryl, and optionally substituted heteroaryl;

R^{b} is chosen from H, optionally substituted C₁-C₆ alkyl, optionally substituted cycloalkyl, optionally substituted heterocycloalkyl, optionally substituted aryl, and optionally substituted heteroaryl; and

R^{c} is independently chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for cycloalkyl, heterocycloalkyl, or heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl); and

wherein optionally substituted acyl, optionally substituted alkoxycarbonyl, sulfinyl and sulfonyl are as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

Compounds of Formula I include, but are not limited to, optical isomers of compounds of Formula I, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I include Z- and E-forms (or cis- and trans-forms) of compounds with carbon-carbon double bonds. Where compounds of Formula I exists in various tautomeric forms, chemical entities of the present invention include all tautomeric forms of the compound.

Chemical entities of the present invention include, but are not limited to compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, chelates, non-covalent complexes, prodrugs, and mixtures.

“Pharmaceutically acceptable salts” include, but are not limited to salts with inorganic acids, such as hydrochloride, phosphate, diphosphate, hydrobromide, sulfate, sulfinate, nitrate, and like salts; as well as salts with an organic acid, such as malate, maleate, fumarate, tartrate, succinate, citrate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compound of Formula I is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, phosphate, and benzoate and like derivatives of functional groups (such as alcohol or amine groups) in the compounds of Formula I.

The term “solvate” refers to the chemical entity formed by the interaction of a solvent and a compound. Suitable solvates are pharmaceutically acceptable solvates, such as hydrates, including monohydrates and hemi-hydrates.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

The term “active agent” is used to indicate a chemical entity which has biological activity. In certain embodiments, an “active agent” is a compound having pharmaceutical utility. For example an active agent may be an anti-cancer therapeutic.

The term “therapeutically effective amount” of a chemical entity of this invention means an amount effective, when administered to a human or non-human patient, to provide a therapeutic benefit such as amelioration of symptoms, slowing of disease progression, or prevention of disease. In some embodiments, a therapeutically effective amount is an amount sufficient to reduce cancer symptoms.

The term “inhibition” indicates a significant decrease in the baseline activity of a biological activity or process.

By “significant” is meant any detectable change that is statistically significant in a standard parametric test of statistical significance such as Student's T-test, where p<0.05.

“Treatment” or “treating” means any treatment of a disease in a patient, including:
a) preventing the disease, that is, causing the clinical symptoms of the disease not to develop;
b) inhibiting the disease;
c) slowing or arresting the development of clinical symptoms; and/or
d) relieving the disease, that is, causing the regression of clinical symptoms.

“Patient” refers to an animal, such as a mammal, that has been or will be the object of treatment, observation or experiment. The methods of the invention can be useful in both human therapy and veterinary applications. In some embodiments, the patient is a mammal; in some embodiments the patient is human; and in some embodiments the patient is chosen from cats and dogs.

The present invention is directed to a class of novel compounds that cause mitotic arrest and cell death for the treatment of disorders associated with cell proliferation.

The compounds, compositions and methods described herein can differ in their selectivity and are used to treat diseases of cellular proliferation, including, but not limited to cancer, hyperplasias, restenosis, cardiac hypertrophy, immune disorders, fungal disorders and inflammation.

Provided is at least one chemical entity chosen from compounds of Formula I 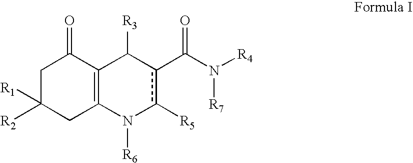
and pharmaceutically acceptable salts, solvates, chelates, non-covalent complexes, prodrugs, and mixtures thereof, wherein
R₁ and R₂ are independently selected from hydrogen, optionally substituted lower alkyl, optionally substituted cycloalkyl, optionally substituted heterocycloalkyl, optionally substituted aryl, and optionally substituted heteroaryl, or wherein R₁ taken together with R₂ form an optionally substituted 3- to 7-membered ring which optionally includes one or two heteroatoms chosen from O, N, and S;
R₃ is selected from optionally substituted aryl and optionally substituted heteroaryl;
R₄ is selected from hydrogen, optionally substituted lower alkyl, optionally substituted aryl and optionally substituted heteroaryl;
R₅ is chosen from hydrogen, optionally substituted lower alkyl, optionally substituted aryl, and optionally substituted heteroaryl, and
R₆ is chosen from hydrogen and optionally substituted lower alkyl, or R₅ taken together with R₆, and the atoms to which they are bound form an optionally substituted 4 to 7-membered ring which optionally includes one, two, or three heteroatoms chosen from N, O, and S, and
R₇ is chosen from hydrogen and optionally substituted lower alkyl, or
R₄ and R₇, taken together with the nitrogen to which they are bound form an optionally substituted 4 to 7-membered ring which optionally includes one, two, or three heteroatoms chosen from N, O, and S, and
   wherein the dashed line indicates that the bond can be either a single or double bond.

The compounds of Formula I can be named and numbered in the manner (e.g., using ChemDraw AutoNom version 2.1 or using Pipeline Pilot or Nomenclator™ available from ChemInnovation Software, Inc.) described below. For example, the compound: 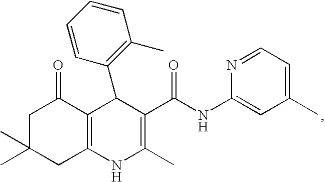
i.e., the compound according to Formula I where R₁ is methyl; R₂ is methyl; R₃ is o-tolyl-; R₄ is 4-methyl-pyridin-2-yl-, R₅ is methyl, R₆ is hydrogen, and R₇ is hydrogen can be named 2,7,7-trimethyl-5-oxo-4-o-tolyl-1,4,5,6,7,8-hexahydro-quinoline-3-carboxylic acid (4-methyl-pyridin-2-yl)-amide.

Likewise, the compound: 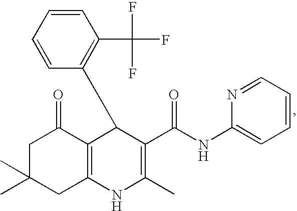
i.e., the compound according to Formula I where R₁ is methyl; R₂ is methyl; R₃ is 2-trifluoromethyl-phenyl-; R₄ is pyridin-2-yl-, R₅ is methyl, R₆ is hydrogen, and R₇ is hydrogen can be named 2,7,7-trimethyl-5-oxo-(2-trifluoromethyl-phenyl)-1,4,5,6,7,8-hexahydro-quinoline-3-carboxylic acid pyridin-2-ylamide.

The compounds of the invention can be synthesized utilizing techniques well known in the art from commercially available starting materials and reagents. For example, the compounds of the invention can be prepared as shown below:

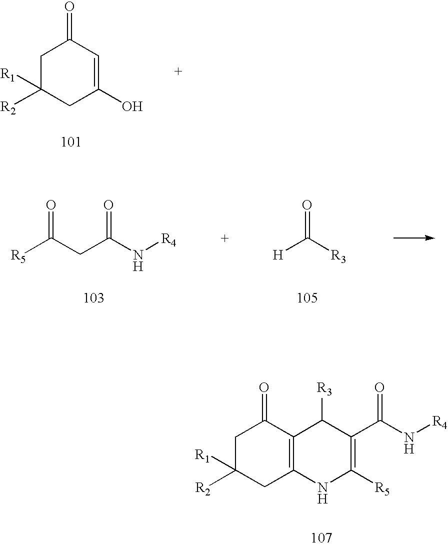

A compound of Formula 101 is combined with a compound of Formula 103 and a compound of Formula 105 with an amine source, such as ammonium acetate in a polar solvent such as methanol. The mixture is refluxed or heated in a microwave reactor at about 130° C. for about 30 min. The product, a compound of Formula 107, is isolated and purified. See, also, Sainani et al. (1994) Indian J. Chem, Sect B: Org. Chem. Including Med. Chem. 33:526-531; Ahluwalla et al. (1996) Indian J. Chem, Sect B, 35:1021-1025; and Simsek, et al. (2000) Farmaco 55:665-668.

Compounds of Formula 103 can be prepared by the treatment of an excess (such as about 3 equivalents) of methyl acetoacetate with an amine of formula R₄NH₂ at about 150° C. for several hours. The product, a compound of Formula 103, is isolated and optionally purified.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures.

When desired, the (R) and (S) isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that when the desired enantiomer is converted into another chemical entity by one of the separation procedures described above, a further step may be required to liberate the desired enantiomeric form. Alternatively, a specific enantiomer may be synthesized by asymmetric synthesis using optically active reagents, substrates, catalysts and/or solvents, or by converting one enantiomer to the other by asymmetric transformation.

A compound of Formula I is optionally contacted with a pharmaceutically acceptable acid or base to form the corresponding acid or base addition salt.

A pharmaceutically acceptable acid addition salt of a compound of Formula I is optionally contacted with a base to form the corresponding free base of Formula I.

A pharmaceutically acceptable base addition salt of a compound of Formula I is optionally contacted with an acid to form the corresponding free acid of Formula I.

In some embodiments, R₁ and R₂ are independently chosen from hydrogen, methyl, and phenyl.

In some embodiments, R₁ is the same as R₂. In some embodiments, R₁ and R₂ are both hydrogen. In some embodiments, R₁ and R₂ are both methyl.

In some embodiments, R₁ taken together with R₂ form an optionally substituted 3- to 7-membered ring.

In some embodiments, R₃ is optionally substituted aryl. In some embodiments, R₃ is optionally substituted phenyl.

In some embodiments, R₃ is aryl or heteroaryl, each of which is optionally substituted with one, two, or three of the following: cyano; optionally substituted amino; halo; hydroxyl; lower alkoxy; optionally substituted lower alkyl; and optionally substituted amino. In some embodiments, R₃ is aryl or heteroaryl, each of which is optionally substituted with one, two or three of the following: cyano, optionally substituted lower alkyl or halo. In some embodiments, R₃ is phenyl optionally substituted with one, two or three groups chosen from cyano, optionally substituted lower alkyl and halo.

In some embodiments, R₃ is chosen from phenyl, 2-methylphenyl, 3-methylphenyl, 4-methylphenyl, 2,3-difluorophenyl, 2,3-dichlorophenyl, 2,4-dichlorophenyl, 3,5-dichlorophenyl, 2,5-dichlorophenyl, 2,6-dichlorophenyl, 3,4-dichlorophenyl, 2-trifluoromethylphenyl, 3-trifluoromethylphenyl, 4-trifluoromethylphenyl, 2-chlorophenyl, 3-chlorophenyl, 4-chlorophenyl, 2-bromophenyl, 3-bromophenyl, 4-bromophenyl, 3-methoxyphenyl, 2-methoxyphenyl, 4-methoxyphenyl, 2-cyanophenyl, 3-cyanophenyl, 4-cyanophenyl, 2,3-dimethylphenyl, 2-ethylphenyl, 2-fluorophenyl, 2-chloro-3-methylphenyl, 3-chloro-2-methylphenyl, 3-fluoro-2-methylphenyl, 3-cyano-2-methylphenyl, and 2,6-dimethylphenyl.

In some embodiments, R₄ is selected from optionally substituted aryl and optionally substituted heteroaryl. In some embodiments, R₄ is optionally substituted phenyl, optionally substituted pyridinyl, optionally substituted imidazolyl, optionally substituted indolyl, optionally substituted benzoimidazolyl, optionally substituted 1H-pyrazolyl, optionally substituted isoxazolyl, or optionally substituted quinolinyl.

In some embodiments, R₄ is aryl or heteroaryl, each of which is optionally substituted with one, two or three of the following: cyano; optionally substituted amino; halo; hydroxyl; lower alkoxy; optionally substituted lower alkyl; and optionally substituted amino. In some embodiments, R₄ is aryl or heteroaryl, each of which is optionally substituted with one, two or three of the following: lower alkyl or halo. In some embodiments, R₄ is pyridinyl or phenyl, each of which is optionally substituted with one, two or three of the following: lower alkyl or halo. In some embodiments, R₄ is pyridin-2-yl or phenyl, each of which is optionally substituted with one, two or three of the following: lower alkyl or halo. In some embodiments, R₄ is 4-methyl-pyridin-2-yl, 3-methyl-pyridin-2-yl, 5-methyl-pyridin-2-yl, pyridin-2-yl, pyridin-3-yl, 6-methyl-pyridin-2-yl, or 4-fluorophenyl.

In some embodiments, R₅ is selected from hydrogen and optionally substituted lower alkyl. In some embodiments, R₅ is optionally substituted lower alkyl. In some embodiments, R₅ is lower alkyl. In some embodiments, R₅ is methyl.

In some embodiments, R₆ is hydrogen.

In some embodiments, R₅ taken together with R₆, and the atoms to which they are bound form an optionally substituted 4 to 7-membered ring which optionally includes one, two, or three heteroatoms chosen from N, O, and S. In some embodiments, a 5 or 6 membered ring is formed.

In some embodiments, R₇ is hydrogen.

In some embodiments, R₄ and R₇, taken together with the nitrogen to which they are bound form an optionally substituted 4 to 7-membered ring which optionally includes one, two, or three heteroatoms chosen from N, O, and S. In some embodiments, a ring chosen from optionally substituted pyrrolidinyl, optionally substituted piperidinyl, optionally substituted piperazinyl, and optionally substituted morpholinyl is formed.

In some embodiments, the compound of Formula I is chosen from compounds of Formula II 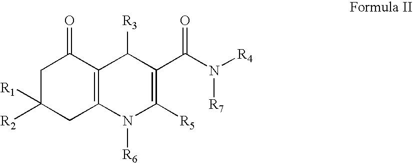
wherein R₁, R₂, R₃, R₄, R₅, R₆, and R₇ are as described for compounds of Formula I.

In some embodiments, the compound of Formula I is chosen from
2,7,7-trimethyl-5-oxo-4-o-tolyl-1,4,5,6,7,8-hexahydro-quinoline-3-carboxylic acid (4-methyl-pyridin-2-yl)-amide;
4-(2,3-dichloro-phenyl)-2-methyl-5-oxo-7-phenyl-1,4,5,6,7,8-hexahydro-quinoline-3-carboxylic acid (3-methyl-pyridin-2-yl)-amide;
2,7,7-trimethyl-5-oxo-4-o-tolyl-1,4,5,6,7,8-hexahydro-quinoline-3-carboxylic acid pyridin-2-ylamide;
2,7,7-trimethyl-5-oxo-4-(2-trifluoromethyl-phenyl)-1,4,5,6,7,8-hexahydro-quinoline-3-carboxylic acid (4-methyl-pyridin-2-yl)-amide;
4-(2-chloro-phenyl)-2,7,7-trimethyl-5-oxo-1,4,5,6,7,8-hexahydro-quinoline-3-carboxylic acid pyridin-2-ylamide;
2,7,7-trimethyl-5-oxo-4-(2-trifluoromethyl-phenyl)-1,4,5,6,7,8-hexahydro-quinoline-3-carboxylic acid pyridin-2-ylamide;
4-(2-chloro-phenyl)-2,7,7-trimethyl-5-oxo-1,4,5,6,7,8-hexahydro-quinoline-3-carboxylic acid (6-methyl-pyridin-2-yl)-amide;
4-(3-bromo-phenyl)-2,7,7-trimethyl-5-oxo-1,4,5,6,7,8-hexahydro-quinoline-3-carboxylic acid (4-methyl-pyridin-2-yl)-amide;
2-methyl-5-oxo-4-o-tolyl-1,4,5,6,7,8-hexahydro-quinoline-3-carboxylic acid (4-fluoro-phenyl)-amide;
4-(2,3-dichloro-phenyl)-2-methyl-5-oxo-1,4,5,6,7,8-hexahydro-quinoline-3-carboxylic acid pyridin-2-ylamide; and
4-(2,3-dichloro-phenyl)-2-methyl-5-oxo-1,4,5,6,7,8-hexahydro-quinoline-3-carboxylic acid (3-methyl-pyridin-2-yl)-amide;
[4-(3-methoxyphenyl)-2,7,7-trimethyl-5-oxo(3-1,4,6,7,8-pentahydroquinolyl)]-N-(4-methyl(2-pyridyl))carboxamide;
[4-(2-methoxyphenyl)-2,7,7-trimethyl-5-oxo(3-1,4,6,7,8-pentahydroquinolyl)]-N-(5-methyl(2-pyridyl))carboxamide;
[4-(2-methoxyphenyl)-2,7,7-trimethyl-5-oxo(3-1,4,6,7,8-pentahydroquinolyl)]-N-(4-methyl(2-pyridyl))carboxamide;
N-(2-pyridyl)[2,7,7-trimethyl-4-(2-methylphenyl)-5-oxo(3-1,4,6,7,8-pentahydroquinolyl)]carboxamide;
N-(5-methyl(2-pyridyl))[2,7,7-trimethyl-4-(2-methylphenyl)-5-oxo(3-1,4,6,7,8-pentahydroquinolyl)]carboxamide;
N-(4-methyl(2-pyridyl))[2,7,7-trimethyl-4-(2-methylphenyl)-5-oxo(3-1,4,6,7,8-pentahydroquinolyl)]carboxamide;
N-(4-methyl(2-pyridyl))[2,7,7-trimethyl-4-(4-methylphenyl)-5-oxo(3-1,4,6,7,8-pentahydroquinolyl)]carboxamide;
[4-(4-chlorophenyl)-2,7,7-trimethyl-5-oxo(3-1,4,6,7,8-pentahydroquinolyl)]-N-(4-methyl(2-pyridyl))carboxamide;
[4-(4-methoxyphenyl)-2,7,7-trimethyl-5-oxo(3-1,4,6,7,8-pentahydroquinolyl)]-N-(4-methyl(2-pyridyl))carboxamide;
[4-(3-chlorophenyl)-2,7,7-trimethyl-5-oxo(3-1,4,6,7,8-pentahydroquinolyl)]-N-(4-methyl(2-pyridyl))carboxamide;
N-(6-methyl(2-pyridyl))(2,7,7-trimethyl-5-oxo-4-(3-pyridyl)(3-1,4,6,7,8-pentahydroquinolyl))carboxamide;
N-(6-methyl(2-pyridyl))(2,7,7-trimethyl-5-oxo-4-(2-thienyl)(3-1,4,6,7,8-pentahydroquinolyl))carboxamide;
[4-(2,3-dichlorophenyl)-2,7,7-trimethyl-5-oxo(3-1,4,6,7,8-pentahydroquinolyl)]-N-(2-pyridyl)carboxamide;
[4-(2,3-dichlorophenyl)-2,7,7-trimethyl-5-oxo(3-1,4,6,7,8-pentahydroquinolyl)]-N-(4-methyl(2-pyridyl))carboxamide;
[4-(2,4-dichlorophenyl)-2,7,7-trimethyl-5-oxo(3-1,4,6,7,8-pentahydroquinolyl)]-N-(4-methyl(2-pyridyl))carboxamide;
N-phenyl(2,7,7-trimethyl-5-oxo-4-phenyl(3-1,4,6,7,8-pentahydroquinolyl))carboxamide;
N-(5-methyl(2-pyridyl)){2,7,7-trimethyl-5-oxo-4-[3-(trifluoromethyl)phenyl](3-1,4,6,7,8-pentahydroquinolyl)}carboxamide
N-(5-methyl(2-pyridyl))[2,7,7-trimethyl-4-(3-methylphenyl)-5-oxo(3-1,4,6,7,8-pentahydroquinolyl)]carboxamide;
[4-(2,3-dichlorophenyl)-2,7,7-trimethyl-5-oxo(3-1,4,6,7,8-pentahydroquinolyl)]-N-(5-methyl(2-pyridyl))carboxamide;
[4-(2,3-dichlorophenyl)-1-(2-hydroxyethyl)-2,7,7-trimethyl-5-oxo(3-1,4,6,7,8-pentahydroquinolyl)]-N-(5-methyl(2-pyridyl))carboxamide;
N-phenyl(2,7,7-trimethyl-5-oxo-4-phenyl(3-1,2,3,4,6,7,8-heptahydroquinolyl))carboxamide;
N-(4-methyl(2-pyridyl)) {2,7,7-trimethyl-5-oxo-4-[3-(trifluoromethyl)phenyl](3-1,4,6,7,8-pentahydroquinolyl)}carboxamide;
N-(4-methyl(2-pyridyl))[2,7,7-trimethyl-4-(3-methylphenyl)-5-oxo(3-1,4,6,7,8-pentahydroquinolyl)]carboxamide;
[4-(2-chlorophenyl)-2,7,7-trimethyl-5-oxo(3-1,4,6,7,8-pentahydroquinolyl)]-N-(4-methyl(2-pyridyl))carboxamide;
[4-(3-cyanophenyl)-2,7,7-trimethyl-5-oxo(3-1,4,6,7,8-pentahydroquinolyl)]-N-(4-methyl(2-pyridyl))carboxamide;
[4-(4-cyanophenyl)-2,7,7-trimethyl-5-oxo(3-1,4,6,7,8-pentahydroquinolyl)]-N-(4-methyl(2-pyridyl))carboxamide;
[4-(3,4-dichlorophenyl)-2,7,7-trimethyl-5-oxo(3-1,4,6,7,8-pentahydroquinolyl)]-N-(4-methyl(2-pyridyl))carboxamide;
[4-(2,3-difluorophenyl)-2,7,7-trimethyl-5-oxo(3-1,4,6,7,8-pentahydroquinolyl)]-N-(4-methyl(2-pyridyl))carboxamide;
[4-(2,3-dimethylphenyl)-2,7,7-trimethyl-5-oxo(3-1,4,6,7,8-pentahydroquinolyl)]-N-(4-methyl(2-pyridyl))carboxamide;
[4-(2-fluorophenyl)-2,7,7-trimethyl-5-oxo(3-1,4,6,7,8-pentahydroquinolyl)]-N-(4-methyl(2-pyridyl))carboxamide;
[4-(3,5-dichlorophenyl)-2,7,7-trimethyl-5-oxo(3-1,4,6,7,8-pentahydroquinolyl)]-N-(4-methyl(2-pyridyl))carboxamide;
[4-(2,5-dichlorophenyl)-2,7,7-trimethyl-5-oxo(3-1,4,6,7,8-pentahydroquinolyl)]-N-(4-methyl(2-pyridyl))carboxamide;
[4-(2,6-dichlorophenyl)-2,7,7-trimethyl-5-oxo(3-1,4,6,7,8-pentahydroquinolyl)]-N-(4-methyl(2-pyridyl))carboxamide;
[4-(2-ethylphenyl)-2,7,7-trimethyl-5-oxo(3-1,4,6,7,8-pentahydroquinolyl)]-N-(4-methyl(2-pyridyl))carboxamide;
[4-(2,3-dichlorophenyl)-2,7,7-trimethyl-5-oxo(3-1,4,6,7,8-pentahydroquinolyl)]-N-benzamide;
[4-(2,6-dimethylphenyl)-2,7,7-trimethyl-5-oxo(3-1,4,6,7,8-pentahydroquinolyl)]-N-(4-methyl(2-pyridyl))carboxamide;
N-(4-methyl(2-pyridyl))(2,7,7-trimethyl-5-oxo-4-(2-pyridyl)(3-1,4,6,7,8-pentahydroquinolyl))carboxamide;
[4-(2,3-dichlorophenyl)-2,7,7-trimethyl-5-oxo(3-1,4,6,7,8-pentahydroquinolyl)]-N-methylcarboxamide;
[4-(2,3-dichlorophenyl)-2,7,7-trimethyl-5-oxo(3-1,4,6,7,8-pentahydroquinolyl)]-N-isoxazol-3-ylcarboxamide;
4-(2,3-dichlorophenyl)-2,7,7-trimethyl-5-oxo-1,4,6,7,8-pentahydroquinoline-3-carboxamide;
[4-(2,3-dichlorophenyl)-2,7,7-trimethyl-5-oxo(3-1,4,6,7,8-pentahydroquinolyl)]-N-(3-pyridyl)carboxamide;
9-(2,3-dichlorophenyl)-3,3,6,6-tetramethyl-2,3,4,5,6,7,9,10-octahydroacridine-1,8-dione;
[4-(2,3-dichlorophenyl)-2,7,7-trimethyl-5-oxo(3-1,4,6,7,8-pentahydroquinolyl)]-N-(1-methylpyrazol-3-yl)carboxamide; and
[4-(2,3-dichlorophenyl)-2,7,7-trimethyl-5-oxo(3-1,4,6,7,8-pentahydroquinolyl)]-N-methyl-N-(4-methyl(2-pyridyl))carboxamide.

The chemical entities described herein are used to treat cellular proliferation diseases. Such disease states include, but are not limited to, cancer (further discussed below), autoimmune disease, fungal disorders, arthritis, graft rejection, inflammatory bowel disease, cellular proliferation induced after medical procedures, including, but not limited to, surgery, angioplasty, and the like. Treatment includes inhibiting cellular proliferation. It is appreciated that in some cases the cells may not be in an abnormal state and still require treatment. Thus, in some embodiments, the chemical entities described herein are applied to cells or individuals afflicted or subject to impending affliction with any one of these disorders or states.

The chemical entities, compositions and methods provided herein are deemed particularly useful for the treatment of cancer including solid tumors such as skin, breast, brain, cervical carcinomas, testicular carcinomas, etc. More particularly, cancers that can be treated include, but are not limited to:
Cardiac: sarcoma (angiosarcoma, fibrosarcoma, rhabdomyosarcoma, liposarcoma), myxoma, rhabdomyoma, fibroma, lipoma and teratoma;
Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver: hepatoma (hepatocellular carcinoma), cholangiocarcinoma, hepatoblastoma, angiosarcoma, hepatocellular adenoma, hemangioma;
Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant tertoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma);
Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];
Skin: malignant melanoma, basal cell carcinoma, squamous cell carcinoma, Karposi's sarcoma, moles dysplastic nevi, lipoma, angioma, dermatofibroma, keloids, psoriasis; and
Adrenal glands: neuroblastoma.

As used herein, treatment of cancer includes treatment of cancerous cells, including cells afflicted by any one of the above-identified conditions. Thus, the term “cancerous cell” as provided herein, includes a cell afflicted by any one of the above identified conditions.

Another useful aspect of the invention is a kit having at least one chemical entity described herein and a package insert or other labeling including directions treating a cellular proliferative disease by administering an effective amount of the at least one chemical entity. In some embodiments, the at least one chemical entity in the kits of the invention is provided as one or more doses for a course of treatment for a cellular proliferative disease, each dose being a pharmaceutical formulation including a pharmaceutical excipient and the at least one chemical entity.

The chemical entities described herein may be demonstrated to inhibit tumor cell proliferation, cell transformation and tumorigenesis in vitro and in vivo using a variety of assays known in the art, or described herein. Such assays may use cells of a cancer cell line, or cells from a patient. Many assays well-known in the art can be used to assess such survival and/or growth; for example, cell proliferation can be assayed by measuring ³H-thymidine incorporation, by direct cell count, by detecting changes in transcription, translation or activity of known genes such as proto-oncogenes (e.g., fos, myc) or cell cycle markers (R^{b}, cdc2, cyclin A, D1, D2, D3, E, etc). The levels of such protein and mRNA and activity can be determined by any method well known in the art.

The present invention provides for cell cycle and cell proliferation analysis by a variety of techniques known in the art. For example, cell proliferation may be measured by counting samples of a cell population over time (e.g., daily cell counts). Cells may be counted using a hemacytometer and light microscopy (e.g., HyLite hemacytometer, Hausser Scientific). Cell number may be plotted against time in order to obtain a growth curve for the population of interest. In some embodiments, cells are first mixed with the dye Trypan-blue (Sigma), such that living cells exclude the dye, and are counted as viable members of the population.

DNA content and/or mitotic index of the cells may be measured, for example, based on DNA ploidy value of the cell. For example, cells in the G1 phase of the cell cycle generally contain a 2N DNA ploidy value. Cells in which DNA has been replicated but have not progressed through mitosis (e.g., cells in S-phase) will exhibit a ploidy value higher than 2N and up to 4N DNA content. Ploidy value and cell-cycle kinetics may be further measured using propidum iodide assay (see, e.g., Turner, T., et al., 1998, Prostate 34:175-81). Alternatively, the DNA ploidy maybe determined by quantitation of DNA Feulgen staining (which binds to DNA in a stoichiometric manner) on a computerized microdensitometrystaining system (see, e.g., Bacus, S., 1989, Am. J. Pathol. 135:783-92). In some embodiments, DNA content may be analyzed by preparation of a chromosomal spread (Zabalou, S., 1994, Hereditas. 120:127-40; Pardue, M. L., 1994, Meth. Cell Biol. 44:333-351).

Detection of changes in length of the cell cycle or speed of cell cycle may also be used to measure inhibition of cell proliferation by the chemical entities described herein. In some embodiments the length of the cell cycle is determined by the doubling time of a population of cells (e.g., using cells contacted or not contacted with at least one chemical entity described herein). In some embodiments, FACS analysis is used to analyze the phase of cell cycle progression, or purify G1, S, and G2/M fractions (see e.g., Delia, D. et al., 1997, Oncogene 14:2137-47).

Lapse of cell cycle checkpoint(s), and/or induction of cell cycle checkpoint(s), may be examined by any method known in the art. Without limitation, a cell cycle checkpoint is a mechanism which ensures that certain cellular events occur in a particular order. Checkpoint genes are defined by mutations that allow late events to occur without prior completion of an early event (Weinert, T., and Hartwell, L., 1993, Genetics 134:63-80). Induction or inhibition of cell cycle checkpoint genes may be assayed, for example, by Western blot analysis, or by immunostaining, etc. Lapse of cell cycle checkpoints may be further assessed by the progression of a cell through the checkpoint without prior occurrence of specific events (e.g., progression into mitosis without complete replication of the genomic DNA).

The chemical entities described herein can also be demonstrated to alter cell proliferation in cultured cells in vitro using methods that are well known in the art. Specific examples of cell culture models include, but are not limited to, for lung cancer, primary rat lung tumor cells (Swafford et al., 1997, Mol. Cell. Biol. 17:1366-1374) and large-cell undifferentiated cancer cell lines (Mabry et al., 1991, Cancer Cells 3:53-58); colorectal cell lines for colon cancer (Park and Gazdar, 1996, J. Cell Biochem. Suppl. 24:131-141); multiple established cell lines for breast cancer (Hambly et al., 1997, Breast Cancer Res. Treat. 43:247-258; Gierthy et al., 1997, Chemosphere 34:1495-1505; Prasad and Church, 1997, Biochem. Biophys. Res. Commun. 232:14-19); a number of well-characterized cell models for prostate cancer (Webber et al., 1996, Prostate, Part 1, 29:386-394; Part 2, 30:58-64; and Part 3, 30-136-142; Boulikas, 1997, Anticancer Res. 17:1471-1505); for genitourinary cancers, continuous human bladder cancer cell lines (Ribeiro et al., 1997, Int. J. Radiat. Biol. 72:11-20); organ cultures of transitional cell carcinomas (Booth et al., 1997, Lab Invest. 76:843-857) and rat progression models (Vet et al., 1997, Biochim. Biophys Acta 1360:39-44); and established cell lines for leukemias and lymphomas (Drexler, 1994, Leuk. Res. 18:919-927; Tohyama, 1997, Int. J. Hematol. 65:309-317).

The chemical entities described herein can also be demonstrated to inhibit cell growth (or mitosis) in vitro. In some embodiments, cells are contacted with at least one chemical entity described herein and examined for lethal phenotype.

The chemical entities described herein can also be demonstrated to inhibit tumor formation in vivo. A vast number of animal models of hyperproliferative disorders, including tumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, New York, p. 1814; and Lovejoy et al., 1997, J. Pathol. 181:130-135). Specific examples include for lung cancer, transplantation of tumor nodules into rats (Wang et al., 1997, Ann. Thorac. Surg. 64:216-219) or establishment of lung cancer metastases in SCID mice depleted of NK cells (Yono and Sone, 1997, Gan To Kagaku Ryoho 24:489-494); for colon cancer, colon cancer transplantation of human colon cancer cells into nude mice (Gutman and Fidler, 1995, World J. Surg. 19:226-234), the cotton top tamarin model of human ulcerative colitis (Warren, 1996, Aliment. Pharmacol. Ther. Supp 12:45-47) and mouse models with mutations of the adenomatous polyposis tumor suppressor (Polakis, 1997, Biochim. Biophys. Acta 1332:F127-F147); for breast cancer, transgenic models of breast cancer (Dankfort and Muller, 1996, Cancer Treat. Res. 83:71-88; Amundadittir et al., 1996, Breast Cancer Res. Treat. 39:119-135) and chemical induction of tumors in rats (Russo and Russo, 1996, Breast Cancer Res. Treat. 39:7-20); for prostate cancer, chemically-induced and transgenic rodent models, and human xenograft models (Royai et al., 1996, Semin. Oncol. 23:35-40); for genitourinary cancers, induced bladder neoplasm in rats and mice (Oyasu, 1995, Food Chem. Toxicol. 33:747-755) and xenografts of human transitional cell carcinomas into nude rats (Jarrett et al., 1995, J. Endourol. 9:1-7); and for hematopoietic cancers, transplanted allogenic marrow in animals (Appelbaum, 1997, Leukemia 11(Suppl. 4):S15-S17). Further, general animal models applicable to many types of cancer have been described, including, but not restricted to, the p53-deficient mouse model (Donehower, 1996, Semin. Cancer Biol. 7:269-278), the Min mouse (Shoemaker et al., 1997, Biochem. Biophys. Acta, 1332:F25-F48), and immune responses to tumors in rat (Frey, 1997, Methods, 12:173-188).

For example, at least one chemical entity described herein can be administered to a test animal, preferably a test animal predisposed to develop a type of tumor, and the test animal subsequently examined for a decreased incidence of tumor formation in comparison with controls not administered the compound. Alternatively, at least one chemical entity described herein can be administered to test animals having tumors (e.g., animals in which tumors have been induced by introduction of malignant, neoplastic, or transformed cells, or by administration of a carcinogen) and subsequently examining the tumors in the test animals for tumor regression in comparison to controls not administered the compound.

One measure of inhibition is IC₅₀, defined as the concentration of the chemical entity at which activity is decreased by fifty percent relative to a control. In some embodiments, the chemical entities described herein have IC₅₀'s of less than about 1 mM; in other embodiments, the chemical entities described herein have IC₅₀'s of less than about 100 μM, less than about 10 μM, less than about 1 μM, less than about 100 nM, or less than about 10 nM. Measurement of IC₅₀ is done using standard assays such as that described herein.

Another measure of inhibition is GI₅₀, defined as the concentration of the compound that results in a decrease in the rate of cell growth by fifty percent. In some embodiments, the chemical entities described herein have GI₅₀'s of less than about 1 mM; alternatively, the chemical entities described herein have a GI₅₀ of less than about 20 μM, less than about 10 μM, less than about 1 μM, less than about 100 nM, or less than about 10 nM. Measurement of GI₅₀ is done using a cell proliferation assay such as described herein. the Chemical entities described herein were found to inhibit cell proliferation.

In vitro potency of small molecule inhibitors is determined, for example, by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete cell kill).

Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation, irrespective of the concentration demonstrating inhibition, have potential clinical usefulness.

Accordingly, the chemical entities described herein are administered to cells. By “administered” herein is meant administration of a therapeutically effective dose of at least one chemical entity described herein to a cell either in a cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

The chemical entities described herein may be administered, especially as a pharmaceutically acceptable composition comprising a pharmaceutical excipient, to a patient, as described herein. Depending upon the manner of introduction, the chemical entities described herein may be formulated in a variety of ways as discussed below. The concentration of therapeutically active compound in the formation may vary from about 0.1-10 wt. %.

The agents may be administered alone or in combination with other treatments, i.e., radiation, or other chemotherapeutic agents such as the taxane class of agents that appear to act on microtubule formation or the camptothecin class of topoisomerase I inhibitors. When used, other chemotherapeutic agents may be administered before, concurrently, or after administration of the chemical entities described herein. In some embodiments, at least one chemical entity described herein is co-administered with one or more other chemotherapeutic agents. By “co-administer” it is meant that the chemical entities are administered to a patient such that the chemical entities as well as the co-administered compound may be found in the patient's bloodstream at the same time, regardless of when the compounds are actually administered, including simultaneously.

The administration of the chemical entities described herein can be done in a variety of ways, including, but not limited to, orally, subcutaneously, intravenously, intranasally, transdermally, intraperitoneally, intramuscularly, intrapulmonarily, vaginally, rectally, or intraocularly. In some instances, the chemical entities described herein may be directly applied as a solution or spray.

Pharmaceutical dosage forms include at least one chemical entity described herein and one or more pharmaceutical excipients. As is known in the art, pharmaceutical excipients are secondary ingredients which function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms; and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance; to ease handling and administration of dose; for convenience of use; or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995), each of which is incorporated herein by reference for all purposes.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colors, flavors, sweetening agents, polymers, waxes or other solubility-retarding materials.

Compositions for intravenous administration will generally comprise intravenous fluids, i.e., sterile solutions of simple chemicals such as sugars, amino acids or electrolytes, which can be easily carried by the circulatory system and assimilated. Such fluids are prepared with water for injection USP.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [full citation previously provided], and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol, e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., 1/6 M; and
sterile water for injection.
   The pH of such fluids may vary, and will typically be from 3.5 to 8 as known in the art.

The chemical entities described herein can be administered alone or in combination with other treatments, e.g., radiation, or other therapeutic agents, such as the taxane class of agents that appear to act on microtubule formation or the camptothecin class of topoisomerase I inhibitors. When so used, other therapeutic agents can be administered before, concurrently (whether in separate dosage forms or in a combined dosage form), or after administration of an active agent of the present invention.

The following examples serve to more fully describe the manner of using the above-described invention. It is understood that these examples in no way serve to limit the true scope of this invention, but rather are presented for illustrative purposes.

### EXAMPLE 1

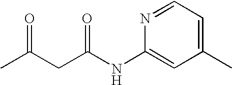

### N-(4-Methyl-pyridin-2-yl)-3-oxo-butyramide

A mixture of methyl acetoacetate (26 mL, 240 mmol) and 2-amino-4-picoline (8.7 g, 80 mmol) was heated at 150° C. under N₂ for 2 h. The reaction mixture was cooled to RT. Recrystallization from 10% CH₂Cl₂/hexanes gave 10 g (65%) of the product as a yellow solid, which turned into a green solid upon standing.

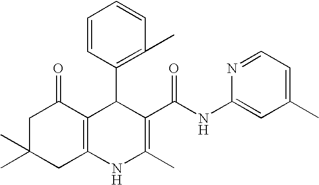

### 2,7,7-Trimethyl-5-oxo-4-o-tolyl-1,4,5,6,7,8-hexahydro-quinoline-3-carboxylic acid (4-methyl-pyridin-2-yl)-amide

A mixture of dimedone (140 mg, 1 mmol), o-tolualdehyde (120 mg, 1 mmol), N-(4-methyl-pyridin-2-yl)-3-oxo-butyramide (192 mg, 1 mmol) and NH₄OAc (112 mg, 1.5 mmol) in methanol (2.5 mL) was heated in a microwave reactor at 130° C. for 30 min. Upon cooling, the yellow precipitate was collected and washed with methanol to 115 mg (28%) of the desired product. LRMS (M-H⁺) m/z 402.4.

### EXAMPLE 2

The following compounds were prepared using procedures similar to those described above.

| Name/MW | M + H |
|---|---|
| [4-(3-methoxyphenyl)-2,7,7-trimethyl-5-oxo(3-1,4,6,7,8-pentahydroquinolyl)]-N-(4- | |
| methyl(2-pyridyl))carboxamide | |
| 431.53 | |
| [4-(2-methoxyphenyl)-2,7,7-trimethyl-5-oxo(3-1,4,6,7,8-pentahydroquinolyl)]-N-(5- | |
| methyl(2-pyridyl))carboxamide | |
| 431.53 | |
| [4-(2-methoxyphenyl)-2,7,7-trimethyl-5-oxo(3-1,4,6,7,8-pentahydroquinolyl)]-N-(4- | 432 |
| methyl(2-pyridyl))carboxamide | |
| 431.53 | |
| N-(2-pyridyl)[2,7,7-trimethyl-4-(2-methylphenyl)-5-oxo(3-1,4,6,7,8- | 402.4 |
| pentahydroquinolyl)]carboxamide | |
| 401.5 | |
| N-(5-methyl(2-pyridyl))[2,7,7-trimethyl-4-(2-methylphenyl)-5-oxo(3-1,4,6,7,8- | |
| pentahydroquinolyl)]carboxamide | |
| 415.53 | |
| N-(4-methyl(2-pyridyl))[2,7,7-trimethyl-4-(2-methylphenyl)-5-oxo(3-1,4,6,7,8- | 416.2 |
| pentahydroquinolyl)]carboxamide | |
| 415.53 | |
| N-(4-methyl(2-pyridyl))[2,7,7-trimethyl-4-(4-methylphenyl)-5-oxo(3-1,4,6,7,8- | 416.2 |
| pentahydroquinolyl)]carboxamide | |
| 415.53 | |
| [4-(4-chlorophenyl)-2,7,7-trimethyl-5-oxo(3-1,4,6,7,8-pentahydroquinolyl)]-N-(4- | 436.2 |
| methyl(2-pyridyl))carboxamide | |
| 435.95 | |
| [4-(4-methoxyphenyl)-2,7,7-trimethyl-5-oxo(3-1,4,6,7,8-pentahydroquinolyl)]-N-(4- | |
| methyl(2-pyridyl))carboxamide | |
| 431.53 | |
| [4-(3-chlorophenyl)-2,7,7-trimethyl-5-oxo(3-1,4,6,7,8-pentahydroquinolyl)]-N-(4- | 436.2 |
| methyl(2-pyridyl))carboxamide | |
| 435.95 | |
| N-(6-methyl(2-pyridyl))(2,7,7-trimethyl-5-oxo-4-(3-pyridyl)(3-1,4,6,7,8- | |
| pentahydroquinolyl))carboxamide | |
| 402.49 | |
| N-(6-methyl(2-pyridyl))(2,7,7-trimethyl-5-oxo-4-(2-thienyl)(3-1,4,6,7,8- | |
| pentahydroquinolyl))carboxamide | |
| 407.53 | |
| [4-(2,3-dichlorophenyl)-2,7,7-trimethyl-5-oxo(3-1,4,6,7,8-pentahydroquinolyl)]-N- | 456 |
| (2-pyridyl)carboxamide | |
| 456.36 | |
| [4-(2,3-dichlorophenyl)-2,7,7-trimethyl-5-oxo(3-1,4,6,7,8-pentahydroquinolyl)]-N- | 470.2 |
| (4-methyl(2-pyridyl))carboxamide | |
| 470.39 | |
| [4-(2,4-dichlorophenyl)-2,7,7-trimethyl-5-oxo(3-1,4,6,7,8-pentahydroquinolyl)]-N- | 470.2 |
| (4-methyl(2-pyridyl))carboxamide | |
| 470.39 | |
| N-phenyl(2,7,7-trimethyl-5-oxo-4-phenyl(3-1,4,6,7,8- | 387.2 |
| pentahydroquinolyl))carboxamide | |
| 386.49 | |
| N-(5-methyl(2-pyridyl)){2,7,7-trimethyl-5-oxo-4-[3-(trifluoromethyl)phenyl](3- | |
| 1,4,6,7,8-pentahydroquinolyl)}carboxamide | |
| 469.5 | |
| N-(5-methyl(2-pyridyl))[2,7,7-trimethyl-4-(3-methylphenyl)-5-oxo(3-1,4,6,7,8- | |
| pentahydroquinolyl)]carboxamide | |
| 415.53 | |
| [4-(2,3-dichlorophenyl)-2,7,7-trimethyl-5-oxo(3-1,4,6,7,8-pentahydroquinolyl)]-N- | |
| (5-methyl(2-pyridyl))carboxamide | |
| 470.39 | |
| [4-(2,3-dichlorophenyl)-1-(2-hydroxyethyl)-2,7,7-trimethyl-5-oxo(3-1,4,6,7,8- | |
| pentahydroquinolyl)]-N-(5-methyl(2-pyridyl))carboxamide | |
| 514.44 | |
| N-phenyl(2,7,7-trimethyl-5-oxo-4-phenyl(3-1,2,3,4,6,7,8- | |
| heptahydroquinolyl))carboxamide | |
| 388.5 | |
| N-(4-methyl(2-pyridyl)){2,7,7-trimethyl-5-oxo-4-[3-(trifluoromethyl)phenyl](3- | 470.2 |
| 1,4,6,7,8-pentahydroquinolyl)}carboxamide | |
| 469.5 | |
| N-(4-methyl(2-pyridyl))[2,7,7-trimethyl-4-(3-methylphenyl)-5-oxo(3-1,4,6,7,8- | 416.2 |
| pentahydroquinolyl)]carboxamide | |
| 415.53 | |
| [4-(2-chlorophenyl)-2,7,7-trimethyl-5-oxo(3-1,4,6,7,8-pentahydroquinolyl)]-N-(4- | 436.2 |
| methyl(2-pyridyl))carboxamide | |
| 435.95 | |
| [4-(3-cyanophenyl)-2,7,7-trimethyl-5-oxo(3-1,4,6,7,8-pentahydroquinolyl)]-N-(4- | |
| methyl(2-pyridyl))carboxamide | |
| 426.51 | |
| [4-(4-cyanophenyl)-2,7,7-trimethyl-5-oxo(3-1,4,6,7,8-pentahydroquinolyl)]-N-(4- | |
| methyl(2-pyridyl))carboxamide | |
| 426.51 | |
| [4-(3,4-dichlorophenyl)-2,7,7-trimethyl-5-oxo(3-1,4,6,7,8-pentahydroquinolyl)]-N- | 470.2 |
| (4-methyl(2-pyridyl))carboxamide | |
| 470.39 | |
| [4-(2,3-difluorophenyl)-2,7,7-trimethyl-5-oxo(3-1,4,6,7,8-pentahydroquinolyl)]-N-(4- | 438.2 |
| methyl(2-pyridyl))carboxamide | |
| 437.48 | |
| [4-(2,3-dimethylphenyl)-2,7,7-trimethyl-5-oxo(3-1,4,6,7,8-pentahydroquinolyl)]-N- | 430.2 |
| (4-methyl(2-pyridyl))carboxamide | |
| 429.55 | |
| [4-(2-fluorophenyl)-2,7,7-trimethyl-5-oxo(3-1,4,6,7,8-pentahydroquinolyl)]-N-(4- | 420.2 |
| methyl(2-pyridyl))carboxamide | |
| 419.49 | |
| [4-(3,5-dichlorophenyl)-2,7,7-trimethyl-5-oxo(3-1,4,6,7,8-pentahydroquinolyl)]-N- | |
| (4-methyl(2-pyridyl))carboxamide | |
| 470.39 | |
| [4-(2,5-dichlorophenyl)-2,7,7-trimethyl-5-oxo(3-1,4,6,7,8-pentahydroquinolyl)]-N- | 470 |
| (4-methyl(2-pyridyl))carboxamide | |
| 470.39 | |
| [4-(2,6-dichlorophenyl)-2,7,7-trimethyl-5-oxo(3-1,4,6,7,8-pentahydroquinolyl)]-N- | 470.2 |
| (4-methyl(2-pyridyl))carboxamide | |
| 470.39 | |
| [4-(2-ethylphenyl)-2,7,7-trimethyl-5-oxo(3-1,4,6,7,8-pentahydroquinolyl)]-N-(4- | 430.2 |
| methyl(2-pyridyl))carboxamide | |
| 429.55 | |
| [4-(2,3-dichlorophenyl)-2,7,7-trimethyl-5-oxo(3-1,4,6,7,8-pentahydroquinolyl)]-N- | 455.2 |
| benzamide | |
| 455.38 | |
| [4-(2,6-dimethylphenyl)-2,7,7-trimethyl-5-oxo(3-1,4,6,7,8-pentahydroquinolyl)]-N- | 430.2 |
| (4-methyl(2-pyridyl))carboxamide | |
| 429.55 | |
| N-(4-methyl(2-pyridyl))(2,7,7-trimethyl-5-oxo-4-(2-pyridyl)(3-1,4,6,7,8- | 403 |
| pentahydroquinolyl))carboxamide | |
| 402.49 | |
| [4-(2,3-dichlorophenyl)-2,7,7-trimethyl-5-oxo(3-1,4,6,7,8-pentahydroquinolyl)]-N- | 393.2 |
| methylcarboxamide | |
| 393.31 | |
| [4-(2,3-dichlorophenyl)-2,7,7-trimethyl-5-oxo(3-1,4,6,7,8-pentahydroquinolyl)]-N- | 446.2 |
| isoxazol-3-ylcarboxamide | |
| 446.33 | |
| 4-(2,3-dichlorophenyl)-2,7,7-trimethyl-5-oxo-1,4,6,7,8-pentahydroquinoline-3- | |
| carboxamide | |
| 379.28 | |
| [4-(2,3-dichlorophenyl)-2,7,7-trimethyl-5-oxo(3-1,4,6,7,8-pentahydroquinolyl)]-N- | 456 |
| (3-pyridyl)carboxamide | |
| 456.36 | |
| 9-(2,3-dichlorophenyl)-3,3,6,6-tetramethyl-2,3,4,5,6,7,9,10-octahydroacridine-1,8- | |
| dione | |
| 418.36 | |
| [4-(2,3-dichlorophenyl)-2,7,7-trimethyl-5-oxo(3-1,4,6,7,8-pentahydroquinolyl)]-N- | 459.2 |
| (1-methylpyrazol-3-yl)carboxamide | |
| 459.37 | |
| [4-(2,3-dichlorophenyl)-2,7,7-trimethyl-5-oxo(3-1,4,6,7,8-pentahydroquinolyl)]-N- | |
| methyl-N-(4-methyl(2-pyridyl))carboxamide | |
| 484.42 | |

### EXAMPLE 3

Inhibition of Cellular Proliferation in Tumor Cell Lines

Cells are plated in 96-well plates at densities from 1000-2500 cells/well of a 96-well plate and allowed to adhere/grow for 24 hours. They are then treated with various concentrations of a compound of the invention for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CeIlTiter 96® AQᵤₑₒᵤₛ One Solution Cell Proliferation Assay) is used to determine the number of viable cells at T₀ and the number of cells remaining after 48 hours compound exposure. The number of cells remaining after 48 hours is compared to the number of viable cells at the time of compound addition, allowing for calculation of growth inhibition.

The growth over 48 hours of cells in control wells that have been treated with vehicle only (0.25% DMSO) is considered 100% growth and the growth of cells in wells with compounds is compared to this.

A GI₅₀ is calculated by plotting the concentration of compound in μM vs the percentage of cell growth in treated wells. The GI₅₀ calculated for the compounds is the estimated concentration at which growth is inhibited by 50% compared to control, i.e., the concentration at which:
100×[(Treated₄₈−T₀)/(Control₄₈−T₀)]=50
wherein Treated₄₈ is the value at 48 hours for the treated cells and Control₄₈ is the value at 48 hours for the control population.

All concentrations of compounds are tested in duplicate and controls are averaged over 12 wells. A very similar 96-well plate layout and GI₅₀ calculation scheme is used by the National Cancer Institute (see Monks et al., 1991, J. Natl. Cancer Inst. 83:757-766). However, the method by which the National Cancer Institute quantitates cell number does not use MTS, but instead employs alternative methods.

While some embodiments have been shown and described, various modifications and substitutions may be made thereto without departing from the spirit and scope of the invention. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present invention has been described by way of illustration and not limitations on the scope of the claims.

1. At least one chemical entity chosen from compounds of Formula I 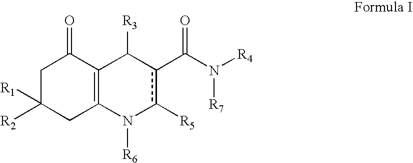
and pharmaceutically acceptable salts, solvates, chelates, non-covalent complexes, prodrugs, and mixtures thereof, wherein
R₁ and R₂ are independently selected from hydrogen, optionally substituted lower alkyl, optionally substituted cycloalkyl, optionally substituted heterocycloalkyl, optionally substituted aryl, and optionally substituted heteroaryl, or wherein R₁ taken together with R₂ form an optionally substituted 3- to 7-membered ring which optionally includes one or two heteroatoms chosen from O, N, and S;
R₃ is selected from optionally substituted aryl and optionally substituted heteroaryl;
R₄ is selected from hydrogen, optionally substituted lower alkyl, optionally substituted aryl and optionally substituted heteroaryl;
R₅ is chosen from hydrogen, optionally substituted lower alkyl, optionally substituted aryl, and optionally substituted heteroaryl;
R₆ is chosen from hydrogen and optionally substituted lower alkyl; or
R₅ taken together with R₆, and the atoms to which they are bound form an optionally substituted 4 to 7-membered ring which optionally includes one, two, or three heteroatoms chosen from N, O, and S, and
R₇ is chosen from hydrogen and optionally substituted lower alkyl, or
wherein R₄ and R₇, taken together with the nitrogen to which they are bound form an optionally substituted 4 to 7-membered ring which optionally includes one, two, or three heteroatoms chosen from N, O, and S.

2. At least one chemical entity of claim 1 wherein the compound of Formula I is chosen from compounds of Formula II

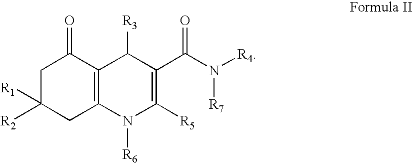

3. At least one chemical entity of claim 1 wherein R₁ and R₂ are independently chosen from hydrogen, methyl, and phenyl.

4. At least one chemical entity of claim 1 wherein R₁ is the same as R₂.

5. At least one chemical entity of claim 4 wherein R₁ and R₂ are both hydrogen.

6. At least one chemical entity of claim 4 wherein R₁ and R₂ are both methyl.

7. At least one chemical entity of claim 1 or 2 wherein R₁ taken together with R₂ form an optionally substituted 3- to 7-membered ring.

8. At least one chemical entity of claim 1 wherein R₃ is optionally substituted aryl.

9. At least one chemical entity of claim 8 wherein R₃ is optionally substituted phenyl.

10. At least one chemical entity of claim 1 wherein R₃ is aryl or heteroaryl, each of which is optionally substituted with one, two, or three groups chosen from cyano; optionally substituted amino; halo; hydroxyl; lower alkoxy; optionally substituted lower alkyl; and optionally substituted amino.

11. At least one chemical entity of claim 10 wherein R₃ is aryl or heteroaryl, each of which is optionally substituted with one, two or three groups independently chosen from cyano, optionally substituted lower alkyl and halo.

12. At least one chemical entity of claim 11 wherein R₃ is phenyl optionally substituted with one, two or three groups independently chosen from cyano, optionally substituted lower alkyl and halo.

13. At least one chemical entity of claim 12 wherein R₃ is chosen from phenyl, 2-methylphenyl, 3-methylphenyl, 4-methylphenyl, 2,3-difluorophenyl, 2,3-dichlorophenyl, 2,4-dichlorophenyl, 3,5-dichlorophenyl, 2,5-dichlorophenyl, 2,6-dichlorophenyl, 3,4-dichlorophenyl, 2-trifluoromethylphenyl, 3-trifluoromethylphenyl, 4-trifluoromethylphenyl, 2-chlorophenyl, 3-chlorophenyl, 4-chlorophenyl, 2-bromophenyl, 3-bromophenyl, 4-bromophenyl, 3-methoxyphenyl, 2-methoxyphenyl, 4-methoxyphenyl, 2-cyanophenyl, 3-cyanophenyl, 4-cyanophenyl, 2,3-dimethylphenyl, 2-ethylphenyl, 2-fluorophenyl, 2-chloro-3-methylphenyl, 3-chloro-2-methylphenyl, 3-fluoro-2-methylphenyl, 3-cyano-2-methylphenyl, and 2,6-dimethylphenyl.

14. At least one chemical entity of claim 1 wherein R₄ is selected from optionally substituted aryl and optionally substituted heteroaryl.

15. At least one chemical entity of claim 14 wherein R₄ is chosen from optionally substituted phenyl, optionally substituted pyridinyl, optionally substituted imidazolyl, optionally substituted indolyl, optionally substituted benzoimidazolyl, optionally substituted 1H-pyrazolyl, optionally substituted isoxazolyl, and optionally substituted quinolinyl.

16. At least one chemical entity of claim 1 wherein R₄ is aryl or heteroaryl, each of which is optionally substituted with one, two or three groups independently chosen from cyano; optionally substituted amino; halo; hydroxyl; lower alkoxy; optionally substituted lower alkyl; and optionally substituted amino.

17. At least one chemical entity of claim 16 wherein R₄ is aryl or heteroaryl, each of which is optionally substituted with one, two or three groups independently chosen from lower alkyl and halo.

18. At least one chemical entity of claim 17 wherein R₄ is pyridinyl or phenyl, each of which is optionally substituted with one, two or three groups independently chosen from lower alkyl and halo.

19. At least one chemical entity of claim 18 wherein R₄ is pyridin-2-yl or phenyl, each of which is optionally substituted with one, two or three groups independently chosen from lower alkyl and halo.

20. At least one chemical entity of claim 19 wherein R₄ is 4-methyl-pyridin-2-yl, 3-methyl-pyridin-2-yl, 5-methyl-pyridin-2-yl, pyridin-2-yl, pyridin-3-yl, 6-methyl-pyridin-2-yl, or 4-fluorophenyl.

21. At least one chemical entity of claim 1 wherein R₅ is selected from hydrogen and optionally substituted lower alkyl.

22. At least one chemical entity of claim 21 wherein R₅ is optionally substituted lower alkyl.

23. At least one chemical entity of claim 22 wherein R₅ is lower alkyl.

24. At least one chemical entity of claim 23 wherein R₅ is methyl.

25. At least one chemical entity of claim 1 wherein R₆ is hydrogen.

26. At least one chemical entity of claim 1 wherein R₇ is hydrogen.

27. At least one chemical entity of claim 1 wherein the compound of Formula I is chosen from
2,7,7-trimethyl-5-oxo-4-o-tolyl-1,4,5,6,7,8-hexahydro-quinoline-3-carboxylic acid (4-methyl-pyridin-2-yl)-amide;
4-(2,3-dichloro-phenyl)-2-methyl-5-oxo-7-phenyl-1,4,5,6,7,8-hexahydro-quinoline-3-carboxylic acid (3-methyl-pyridin-2-yl)-amide;
2,7,7-trimethyl-5-oxo-4-o-tolyl-1,4,5,6,7,8-hexahydro-quinoline-3-carboxylic acid pyridin-2-ylamide;
2,7,7-trimethyl-5-oxo-4-(2-trifluoromethyl-phenyl)-1,4,5,6,7,8-hexahydro-quinoline-3-carboxylic acid (4-methyl-pyridin-2-yl)-amide;
4-(2-chloro-phenyl)-2,7,7-trimethyl-5-oxo-1,4,5,6,7,8-hexahydro-quinoline-3-carboxylic acid pyridin-2-ylamide;
2,7,7-trimethyl-5-oxo-4-(2-trifluoromethyl-phenyl)-1,4,5,6,7,8-hexahydro-quinoline-3-carboxylic acid pyridin-2-ylamide;
4-(2-chloro-phenyl)-2,7,7-trimethyl-5-oxo-1,4,5,6,7,8-hexahydro-quinoline-3-carboxylic acid (6-methyl-pyridin-2-yl)-amide;
4-(3-bromo-phenyl)-2,7,7-trimethyl-5-oxo-1,4,5,6,7,8-hexahydro-quinoline-3-carboxylic acid (4-methyl-pyridin-2-yl)-amide;
2-methyl-5-oxo-4-o-tolyl-1,4,5,6,7,8-hexahydro-quinoline-3-carboxylic acid (4-fluoro-phenyl)-amide;
4-(2,3-dichloro-phenyl)-2-methyl-5-oxo-1,4,5,6,7,8-hexahydro-quinoline-3-carboxylic acid pyridin-2-ylamide; and
4-(2,3-dichloro-phenyl)-2-methyl-5-oxo-1,4,5,6,7,8-hexahydro-quinoline-3-carboxylic acid (3-methyl-pyridin-2-yl)-amide;
[4-(3-methoxyphenyl)-2,7,7-trimethyl-5-oxo(3-1,4,6,7,8-pentahydroquinolyl)]-N-(4-methyl(2-pyridyl))carboxamide;
[4-(2-methoxyphenyl)-2,7,7-trimethyl-5-oxo(3-1,4,6,7,8-pentahydroquinolyl)]-N-(5-methyl(2-pyridyl))carboxamide;
[4-(2-methoxyphenyl)-2,7,7-trimethyl-5-oxo(3-1,4,6,7,8-pentahydroquinolyl)]-N-(4-methyl(2-pyridyl))carboxamide;
N-(2-pyridyl)[2,7,7-trimethyl-4-(2-methylphenyl)-5-oxo(3-1,4,6,7,8-pentahydroquinolyl)]carboxamide;
N-(5-methyl(2-pyridyl))[2,7,7-trimethyl-4-(2-methylphenyl)-5-oxo(3-1,4,6,7,8-pentahydroquinolyl)]carboxamide;
N-(4-methyl(2-pyridyl))[2,7,7-trimethyl-4-(2-methylphenyl)-5-oxo(3-1,4,6,7,8-pentahydroquinolyl)]carboxamide;
N-(4-methyl(2-pyridyl))[2,7,7-trimethyl-4-(4-methylphenyl)-5-oxo(3-1,4,6,7,8-pentahydroquinolyl)]carboxamide;
[4-(4-chlorophenyl)-2,7,7-trimethyl-5-oxo(3-1,4,6,7,8-pentahydroquinolyl)]-N-(4-methyl(2-pyridyl))carboxamide;
[4-(4-methoxyphenyl)-2,7,7-trimethyl-5-oxo(3-1,4,6,7,8-pentahydroquinolyl)]-N-(4-methyl(2-pyridyl))carboxamide;
[4-(3-chlorophenyl)-2,7,7-trimethyl-5-oxo(3-1,4,6,7,8-pentahydroquinolyl)]-N-(4-methyl(2-pyridyl))carboxamide;
N-(6-methyl(2-pyridyl))(2,7,7-trimethyl-5-oxo-4-(3-pyridyl)(3-1,4,6,7,8-pentahydroquinolyl))carboxamide;
N-(6-methyl(2-pyridyl))(2,7,7-trimethyl-5-oxo-4-(2-thienyl)(3-1,4,6,7,8-pentahydroquinolyl))carboxamide;
[4-(2,3-dichlorophenyl)-2,7,7-trimethyl-5-oxo(3-1,4,6,7,8-pentahydroquinolyl)]-N-(2-pyridyl)carboxamide;
[4-(2,3-dichlorophenyl)-2,7,7-trimethyl-5-oxo(3-1,4,6,7,8-pentahydroquinolyl)]-N-(4-methyl(2-pyridyl))carboxamide;
[4-(2,4-dichlorophenyl)-2,7,7-trimethyl-5-oxo(3-1,4,6,7,8-pentahydroquinolyl)]-N-(4-methyl(2-pyridyl))carboxamide;
N-phenyl(2,7,7-trimethyl-5-oxo-4-phenyl(3-1,4,6,7,8-pentahydroquinolyl))carboxamide;
N-(5-methyl(2-pyridyl)){2,7,7-trimethyl-5-oxo-4-[3-(trifluoromethyl)phenyl](3-1,4,6,7,8-pentahydroquinolyl)}carboxamide
N-(5-methyl(2-pyridyl))[2,7,7-trimethyl-4-(3-methylphenyl)-5-oxo(3-1,4,6,7,8-pentahydroquinolyl)]carboxamide;
[4-(2,3-dichlorophenyl)-2,7,7-trimethyl-5-oxo(3-1,4,6,7,8-pentahydroquinolyl)]-N-(5-methyl(2-pyridyl))carboxamide;
[4-(2,3-dichlorophenyl)-1-(2-hydroxyethyl)-2,7,7-trimethyl-5-oxo(3-1,4,6,7,8-pentahydroquinolyl)]-N-(5-methyl(2-pyridyl))carboxamide;
N-phenyl(2,7,7-trimethyl-5-oxo-4-phenyl(3-1,2,3,4,6,7,8-heptahydroquinolyl))carboxamide;
N-(4-methyl(2-pyridyl)){2,7,7-trimethyl-5-oxo-4-[3-(trifluoromethyl)phenyl](3-1,4,6,7,8-pentahydroquinolyl)}carboxamide;
N-(4-methyl(2-pyridyl))[2,7,7-trimethyl-4-(3-methylphenyl)-5-oxo(3-1,4,6,7,8-pentahydroquinolyl)]carboxamide;
[4-(2-chlorophenyl)-2,7,7-trimethyl-5-oxo(3-1,4,6,7,8-pentahydroquinolyl)]-N-(4-methyl(2-pyridyl))carboxamide;
[4-(3-cyanophenyl)-2,7,7-trimethyl-5-oxo(3-1,4,6,7,8-pentahydroquinolyl)]-N-(4-methyl(2-pyridyl))carboxamide;
[4-(4-cyanophenyl)-2,7,7-trimethyl-5-oxo(3-1,4,6,7,8-pentahydroquinolyl)]-N-(4-methyl(2-pyridyl))carboxamide;
[4-(3,4-dichlorophenyl)-2,7,7-trimethyl-5-oxo(3-1,4,6,7,8-pentahydroquinolyl)]-N-(4-methyl(2-pyridyl))carboxamide;
[4-(2,3-difluorophenyl)-2,7,7-trimethyl-5-oxo(3-1,4,6,7,8-pentahydroquinolyl)]-N-(4-methyl(2-pyridyl))carboxamide;
[4-(2,3-dimethylphenyl)-2,7,7-trimethyl-5-oxo(3-1,4,6,7,8-pentahydroquinolyl)]-N-(4-methyl(2-pyridyl))carboxamide;
[4-(2-fluorophenyl)-2,7,7-trimethyl-5-oxo(3-1,4,6,7,8-pentahydroquinolyl)]-N-(4-methyl(2-pyridyl))carboxamide;
[4-(3,5-dichlorophenyl)-2,7,7-trimethyl-5-oxo(3-1,4,6,7,8-pentahydroquinolyl)]-N-(4-methyl(2-pyridyl))carboxamide;
[4-(2,5-dichlorophenyl)-2,7,7-trimethyl-5-oxo(3-1,4,6,7,8-pentahydroquinolyl)]-N-(4-methyl(2-pyridyl))carboxamide;
[4-(2,6-dichlorophenyl)-2,7,7-trimethyl-5-oxo(3-1,4,6,7,8-pentahydroquinolyl)]-N-(4-methyl(2-pyridyl))carboxamide;
[4-(2-ethylphenyl)-2,7,7-trimethyl-5-oxo(3-1,4,6,7,8-pentahydroquinolyl)]-N-(4-methyl(2-pyridyl))carboxamide;
[4-(2,3-dichlorophenyl)-2,7,7-trimethyl-5-oxo(3-1,4,6,7,8-pentahydroquinolyl)]-N-benzamide;
[4-(2,6-dimethylphenyl)-2,7,7-trimethyl-5-oxo(3-1,4,6,7,8-pentahydroquinolyl)]-N-(4-methyl(2-pyridyl))carboxamide;
N-(4-methyl(2-pyridyl))(2,7,7-trimethyl-5-oxo-4-(2-pyridyl)(3-1,4,6,7,8-pentahydroquinolyl))carboxamide;
[4-(2,3-dichlorophenyl)-2,7,7-trimethyl-5-oxo(3-1,4,6,7,8-pentahydroquinolyl)]-N-methylcarboxamide;
[4-(2,3-dichlorophenyl)-2,7,7-trimethyl-5-oxo(3-1,4,6,7,8-pentahydroquinolyl)]-N-isoxazol-3-ylcarboxamide;
4-(2,3-dichlorophenyl)-2,7,7-trimethyl-5-oxo-1,4,6,7,8-pentahydroquinoline-3-carboxamide;
[4-(2,3-dichlorophenyl)-2,7,7-trimethyl-5-oxo(3-1,4,6,7,8-pentahydroquinolyl)]-N-(3-pyridyl)carboxamide;
9-(2,3-dichlorophenyl)-3,3,6,6-tetramethyl-2,3,4,5,6,7,9,10-octahydroacridine-1,8-dione;
[4-(2,3-dichlorophenyl)-2,7,7-trimethyl-5-oxo(3-1,4,6,7,8-pentahydroquinolyl)]-N-(1-methylpyrazol-3-yl)carboxamide; and
[4-(2,3-dichlorophenyl)-2,7,7-trimethyl-5-oxo(3-1,4,6,7,8-pentahydroquinolyl)]-N-methyl-N-(4-methyl(2-pyridyl))carboxamide.

28. A pharmaceutical composition comprising a therapeutically effective amount of at least one chemical entity of claim 1 and one or more pharmaceutical excipients.

29. A method of treating a cellular proliferative disease comprising administering to a patient in need of such treatment at least one chemical entity of claim 1 any one of claims 1 to 27 in a therapeutically effective amount to treat the cellular proliferative disease.

30. The method of claim 29 wherein the cellular proliferative disease is cancer, hyperplasia, restenosis, cardiac hypertrophy, an immune disorder, a fungal disorder, or inflammation.

31. The method of claim 30 wherein the cellular proliferative disease is cancer.

32. (canceled)
